SEQUENCE LISTING (4 sequences)

SEQ ID NO: 1 - DNA, 20 bp, Artificial Sequence
  Forward Primer
taccccatcccaatgactgt

SEQ ID NO: 2 - DNA, 20 bp, Artificial Sequence
  Reverse Primer
atctccccaacatgagtcca

SEQ ID NO: 3 - DNA, 680 bp, Homo sapiens
acctcgcacagcctctcccacaggtaccatgaaggtctccgcggcagccctcgctgtcatcctcattgctactgccctctgcgctcctgcatctgcctccccatgtaagtcctggtcttgaccaccacagcccctggagtcagactctgcctagaatgcctaccccatcccaatgactgtcccacaccttgccctcttttcaactgtagcatgaatactgtctgaaccccattatttacattcttatgaattgtcttacccatatgtcctgttgtccttgagagatctctatgctgcttcatggcagggatctcctgatcagtttttctgtctttaaggtctacaccctcaaggcctacaggtgttcactgagtgtggactcatgttggggagattagtagtcatgagtagctcacactgactttgaaacacgtctgactcatgcctgtcagcagcagggtttctaagaaatttaactaaactaaaacttcctgagaccctgagacagccatattctctctcatttcataaatgtaaaaaaaattggcttatgagagggtatgtggctttattagagtgaatacatttaaaaaacaaataaaaacaaggagaggataataaaagctaattaaatggttactatggatcaggcattgtgctgattttggcacatctttttt

SEQ ID NO: 4 - DNA, 680 bp, Homo sapiens
acctcgcacagcctctcccacaggtaccatgaaggtctccgcggcagccctcgctgtcatcctcattgctactgccctctgcgctcctgcatctgcctccccatgtaagtcctggtcttgaccaccacagcccctggagtcagactctgcctagaatgcctaccccatcccaatgactgtcccacaccttgccctcttttcaactgtagcatgaatactgtctgaaccccattatttacattcttatgaattgtcttacccatatgtcctgttgtccttgagagatctctatgctgcttcatggcagggatctcctgatcagtttttctgtcttcaaggtctacaccctcaaggcctacaggtgttcactgagtgtggactcatgttggggagattagtagtcatgagtagctcacactgactttgaaacacgtctgactcatgcctgtcagcagcagggtttctaagaaatttaactaaactaaaacttcctgagaccctgagacagccatattctctctcatttcataaatgtaaaaaaaattggcttatgagagggtatgtggctttattagagtgaatacatttaaaaaacaaataaaaacaaggagaggataataaaagctaattaaatggttactatggatcaggcattgtgctgattttggcacatctttttt

(19) United States
(12) Patent Application Publication
(10) Pub. No.: US 2018/0340224 A1
(43) Pub. Date: Nov. 29, 2018

(54) METHOD FOR IDENTIFYING A GREATER RISK FOR DEVELOPING BRONCHOPULMONARY DYSPLASIA AND PRIMER PAIR FOR GENOTYPING RANTES GENE SNP AND METHOD THEREOF

(71) Applicant: Meribank Biotech Co., Ltd., New Taipei City (TW)

(72) Inventors: Chang-Yo HSUAN, Taipei City (TW); Willie LIN, Taipei City (TW); Wei-Ting LIU, Taipei City (TW); Meng-Hua LEE, Taipei City (TW); Ting-Ting TSENG, Taipei City (TW)

(73) Assignee: Meribank Biotech Co., Ltd., New Taipei City (TW)

(21) Appl. No.: 15/606,732

(22) Filed: May 26, 2017

(51) Int. Cl. C12Q 1/68

(52) U.S. Cl. CPC C12Q 1/6883; C12Q 2600/118; C12Q 2600/156; C12Q 2600/158

(57) ABSTRACT

Disclosed is a method for identifying a greater risk for developing bronchopulmonary dysplasia (BPD) of a preterm infant. The method comprises obtaining a genomic DNA sample from the preterm infant's mother, genotyping rs2280789 SNP in the RANTES gene, and determining the preterm infant as being at risk of developing BPD if the genotype of the rs2280789 SNP is CC. Also disclosed are a primer pair for genotyping rs2280789 SNP in the RANTES gene, and a method thereof.

### FIELD OF THE INVENTION

The present invention pertains to a method for identifying a greater risk for developing bronchopulmonary dysplasia (BPD) of preterm birth. The present invention also relates to a primer pair for genotyping rs2280789 SNP in the RANTES gene, and a method thereof.

### BACKGROUND OF THE INVENTION

Preterm birth (PTB), or birth before 37 weeks of gestation period, is the major cause of neonatal mortality and morbidity worldwide. Approximately 70% of the neonatal deaths are due to preterm delivery.

Bronchopulmonary dysplasia (BPD), a common chronic inflammatory lung disease of very-low-birth-weight (VLBW) preterm infants, is associated with arrested lung development and treatment of supplemental oxygen [1]. Due to the influences of long-term oxygen therapy and mechanical ventilation, many of these preterm infants consequently acquire different types of problems, such as highly reactive airway diseases, recurrent lower respiratory tract infections, abrupt alveolar development, growth retardation, and feeding difficulties [2, 3]. While early detection of BPD is crucial to prevent chronic symptoms and complications later in life, diagnosis and prevention of this disease remains challenging due to the lack of good biomarkers for identification of infants at risk [1]. It has been reported that interleukin-8 (IL-8) and C-reactive protein (CRP), two recently identified preterm biomarkers, can also be biomarkers for BPD [4, 5].

There is still an urgent need for novel and efficient biomarkers for BPD.

### BRIEF SUMMARY OF THE INVENTION

In one aspect, the present invention provides a method for identifying a greater risk for developing bronchopulmonary dysplasia (BPD) of a preterm infant, comprising obtaining a genomic DNA sample from the preterm infant's mother, genotyping rs2280789 SNP in the RANTES gene, and determining the preterm infant as being at risk of developing BPD if the genotype of the rs2280789 SNP is CC.

According to certain preferred embodiments of the present invention, the method comprises genotyping the rs2280789 SNP using a primer pair comprising a forward primer of SEQ ID NO: 1, and a reverse primer of SEQ ID NO: 2.

In one embodiment of the present invention, the genotype of the rs2280789 SNP is determined by a process comprising performing qPCR using the primer pair to obtain a first melting curve of a first reference sample for the genotype TT, a second melting curve of a second reference sample for the genotype CC, and a third melting curve of the genomic DNA sample; subtracting the first melting curve from each of the first, second and third melting curves to obtain a first, second, and third difference curves, respectively; and comparing the third difference curve with the first and second difference curves, respectively, so as to determine the genotype of the rs2280789 SNP.

In another embodiment, the genotype of the rs2280789 SNP is determined by a process comprising performing qPCR using the primer pair to obtain a plurality of first melting curves of a first reference sample for the genotype TT, a plurality of second melting curves of a second reference sample for the genotype CC, and a plurality of third melting curves of the genomic DNA sample; subtracting the average of the plurality of first melting curves from each of the plurality of first, second and third melting curves to obtain a plurality of first, second, and third difference curves, respectively; and comparing the plurality of third difference curves with the plurality of first and second difference curves, respectively, so as to determine the genotype of the rs2280789 SNP.

In another aspect, the present invention provides a primer pair for genotyping rs2280789 SNP in the RANTES gene, comprising a forward primer of SEQ ID NO: 1, and a reverse primer of SEQ ID NO: 2.

In a further aspect, provided is a method for genotyping rs2280789 SNP in the RANTES gene. The method comprises performing qPCR using a primer pair according to the present invention to obtain a first melting curve of a first reference sample for the genotype TT, a second melting curve of a second reference sample for the genotype CC, and a third melting curve of the genomic DNA sample; subtracting the first melting curve from each of the first, second and third melting curves to obtain a first, second, and third difference curves, respectively; and comparing the third difference curve with the first and second difference curves, respectively, so as to determine the genotype of the rs2280789 SNP.

According to certain preferred embodiments of the present invention, the genotype of the rs2280789 SNP is determined as TT if the third difference curve fits better with the first difference curve than the second difference curve; the genotype of the rs2280789 SNP is determined as CC if the third difference curve fits better with the second difference curve than the first difference curve; and if otherwise, the third difference curve is in between the first difference curve and the second difference curve, the genotype of the rs2280789 SNP is determined as TC.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows the results of gel electrophoresis analysis of PCR products.

FIG. 2 is a difference curve plot. 1: first difference curves, 2: second difference curves, 3: third difference curves.

FIG. 3 is a difference curve plot in connection with sample name TSG010. “TT”: first difference curves, “CC”: second difference curves, “Sample”: third difference curves.

FIG. 4 is a difference curve plot in connection with sample name TSG015. “TT”: first difference curves, “CC”: second difference curves, “Sample”: third difference curves.

FIG. 5 is a difference curve plot in connection with sample name CK016. “TT”: first difference curves, “CC”: second difference curves, “Sample”: third difference curves.

### DETAILED DESCRIPTION OF THE INVENTION

In one aspect, the present invention provides a method for identifying a greater risk for developing bronchopulmonary dysplasia (BPD) of a preterm infant. The method comprises the following steps: obtaining a genomic DNA sample from the preterm infant's mother; genotyping rs2280789 SNP in the RANTES gene (also known as CCL5); and determining the preterm infant as being at risk of developing BPD if the genotype of the rs2280789 SNP is CC.

The term “SNP” as used herein means a single nucleotide polymorphism which is a single nucleotide position in a nucleotide sequence for which two or more alternative alleles are present in a given population.

According to the present invention, the genomic DNA sample may be derived from a tissue selected from the group consisting of blood, placenta, amniotic membrane, chorionic disk, chorionic membrane, and umbilical cord, but is not limited thereto. In one embodiment of the present invention, the blood is umbilical cord blood. In another embodiment, the blood is peripheral blood.

According to certain preferred embodiments of the present invention, the method comprises genotyping the rs2280789 SNP using a primer pair comprising a forward primer of SEQ ID NO: 1 (TACCCCATCCCAATGACTGT), and a reverse primer of SEQ ID NO: 2 (ATCTCCCCAACATGAGTCCA).

In certain embodiments of the present invention, the genotype of the rs2280789 SNP in the RANTES gene of the genomic DNA sample is determined by a process comprising the following steps: (a) performing qPCR using the primer pair to obtain a melting curve of a first reference sample for the genotype TT (the “first melting curve”), a melting curve of a second reference sample for the genotype CC (the “second melting curve”), and a melting curve of the genomic DNA sample (the “third melting curve”); (b) subtracting the first melting curve from each of the first, second and third melting curves to obtain a first, second, and third difference curves, respectively; and (c) comparing the third difference curve with the first and second difference curves, respectively, so as to determine the genotype of the rs2280789 SNP.

According to one embodiment of the present invention, the genotype of the rs2280789 SNP is determined as TT if the third difference curve fits better with the first difference curve than the second difference curve; the genotype of the rs2280789 SNP is determined as CC if the third difference curve fits better with the second difference curve than the first difference curve; and if otherwise, the third difference curve is in between the first difference curve and the second difference curve, the genotype of the rs2280789 SNP is determined as TC.

In some other embodiments, the genotype of the rs2280789 SNP is determined based on an arithmetic mean of SP1/RC1 and SP2/RC2, where SP1 and SP2 are the smallest and largest extreme values, respectively, of the third difference curve, and RC1 and RC2 are the smallest and largest extreme values, respectively, of the second difference curve. A lower arithmetic mean indicates that the genotype is TT, a moderate arithmetic mean indicates that the genotype is TC, and a higher arithmetic mean indicates that the genotype is CC. According to one specific example, the genotype is determined as TT if the arithmetic mean is smaller than 0.26, the genotype is determined as TC if the arithmetic mean is in the range of 0.26 to 1.04, and the genotype is determined as CC if the arithmetic mean is larger than 1.04.

In another embodiment, the genotype of the rs2280789 SNP is determined by a process comprising the following steps: (a) performing qPCR using the primer pair to obtain a plurality of melting curves of a first reference sample for the genotype TT (the “first melting curves”), a plurality of melting curves of a second reference sample for the genotype CC (the “second melting curves”), and a plurality of melting curves of the genomic DNA sample (the “third melting curves”); (b) subtracting the average of the plurality of first melting curves from each of the plurality of first, second and third melting curves to obtain a plurality of first, second, and third difference curves, respectively; and (c) comparing the plurality of third difference curves with the plurality of first and second difference curves, respectively, so as to determine the genotype of the rs2280789 SNP.

According to one embodiment of the present invention, the genotype of the rs2280789 SNP is determined as TT if the plurality of third difference curves fits better with the plurality of first difference curves than the plurality of second difference curves; the genotype of the rs2280789 SNP is determined as CC if the plurality of third difference curves fits better with the plurality of second difference curves than the plurality of first difference curves; and if otherwise, the plurality of third difference curves is in between the plurality of first difference curves and the plurality of second difference curves, genotype of the rs2280789 SNP is determined as TC.

In some other embodiments, the genotype of the rs2280789 SNP is determined based on an arithmetic mean of SP1/RC1 and SP2/RC2, where SP1 and SP2 are the smallest and largest extreme values, respectively, of the third difference curve, and RC1 and RC2 are the smallest and largest extreme values, respectively, of the second difference curve. A lower arithmetic mean indicates that the genotype is TT, a moderate arithmetic mean indicates that the genotype is TC, and a higher arithmetic mean indicates that the genotype is CC. According to one specific example, the genotype is determined as TT if the arithmetic mean is smaller than 0.26, the genotype is determined as TC if the arithmetic mean is in the range of 0.26 to 1.04, and the genotype is determined as CC if the arithmetic mean is larger than 1.04.

In another aspect, the present invention provides a primer pair for genotyping rs2280789 SNP in the RANTES gene, comprising a forward primer of SEQ ID NO: 1, and a reverse primer of SEQ ID NO: 2.

In a further aspect, provided is a method for genotyping rs2280789 SNP in the RANTES gene. The method comprises (a) performing qPCR using a primer pair according to the present invention to obtain a first melting curve of a first reference sample for the genotype TT, a second melting curve of a second reference sample for the genotype CC, and a third melting curve of the genomic DNA sample; (b) subtracting the first melting curve from each of the first, second and third melting curves to obtain a first, second, and third difference curves, respectively; and (c) comparing the third difference curve with the first and second difference curves, respectively, so as to determine the genotype of the rs2280789 SNP.

According to one embodiment of the present invention, the genotype of the rs2280789 SNP is determined as TT if the third difference curve fits better with the first difference curve than the second difference curve; the genotype of the rs2280789 SNP is determined as CC if the third difference curve fits better with the second difference curve than the first difference curve; and if otherwise, the third difference curve is in between the first difference curve and the second difference curve, the genotype of the rs2280789 SNP is determined as TC.

In some other embodiments, the genotype of the rs2280789 SNP is determined based on an arithmetic mean of SP1/RC1 and SP2/RC2, where SP1 and SP2 are the smallest and largest extreme values, respectively, of the third difference curve, and RC1 and RC2 are the smallest and largest extreme values, respectively, of the second difference curve. A lower arithmetic mean indicates that the genotype is TT, a moderate arithmetic mean indicates that the genotype is TC, and a higher arithmetic mean indicates that the genotype is CC. According to one specific example, the genotype is determined as TT if the arithmetic mean is smaller than 0.26, the genotype is determined as TC if the arithmetic mean is in the range of 0.26 to 1.04, and the genotype is determined as CC if the arithmetic mean is larger than 1.04.

In a still further aspect, the present invention provides a method for genotyping rs2280789 SNP in the RANTES gene, comprising (a) performing qPCR using the primer pair to obtain a plurality of melting curves of a first reference sample for the genotype TT (the “first melting curves”), a plurality of melting curves of a second reference sample for the genotype CC (the “second melting curves”), and a plurality of melting curves of the genomic DNA sample (the “third melting curves”); (b) subtracting the average of the plurality of first melting curves from each of the plurality of first, second and third melting curves to obtain a plurality of first, second, and third difference curves, respectively; and (c) comparing the plurality of third difference curves with the plurality of first and second difference curves, respectively, so as to determine the genotype of the rs2280789 SNP.

According to one embodiment of the present invention, the genotype of the rs2280789 SNP is determined as TT if the plurality of third difference curves fits better with the plurality of first difference curves than the plurality of second difference curves; the genotype of the rs2280789 SNP is determined as CC if the plurality of third difference curves fits better with the plurality of second difference curves than the plurality of first difference curves; and if otherwise, the plurality of third difference curves is in between the plurality of first difference curves and the plurality of second difference curves, genotype of the rs2280789 SNP is determined as TC.

In some other embodiments, the genotype of the rs2280789 SNP is determined based on an arithmetic mean of SP1/RC1 and SP2/RC2, where SP1 and SP2 are the smallest and largest extreme values, respectively, of the average of the plurality of first difference curves, and RC1 and RC2 are the smallest and largest extreme values, respectively, of the average of the plurality of second difference curves. A lower arithmetic mean indicates that the genotype is TT, a moderate arithmetic mean indicates that the genotype is TC, and a higher arithmetic mean indicates that the genotype is CC. According to one specific example, the genotype is determined as TT if the arithmetic mean is smaller than 0.26, the genotype is determined as TC if the arithmetic mean is in the range of 0.26 to 1.04, and the genotype is determined as CC if the arithmetic mean is larger than 1.04.

According to the present invention, the first reference sample for the genotype TT and the second reference sample for the genotype CC each may be a synthesized polynucleotide comprising a fragment of the RANTES gene which includes the rs2280789 SNP site (with the nucleotide being a T or C), or a plasmid inserted with such synthesized polynucleotide. In one specific example, the first reference sample is a plasmid inserted with a polynucleotide of SEQ ID NO: 3, and the second reference sample is a plasmid inserted with a polynucleotide of SEQ ID NO: 4.

The present invention is further illustrated by the following examples, which are provided for the purpose of demonstration rather than limitation.

### EXAMPLES

### Example 1: Specificity of the Primer Pair

Perform qPCR using the forward primer of SEQ ID NO: 1 and the reverse primer of SEQ ID NO: 2 on a first reference sample for the genotype TT and a second reference sample for the genotype CC. The first reference sample contains a plasmid inserted with a polynucleotide of SEQ ID NO: 3, the second reference sample contains a plasmid inserted with a polynucleotide of SEQ ID NO: 4. The nucleotide sequence of SEQ ID NO: 3 is that of base 5041 to base 5702 of the RANTES gene (NCBI Reference Sequence: NG_015990.1) where base 5375 is a T, and the nucleotide sequence of SEQ ID NO: 4 is that of base 5041 to base 5702 of the RANTES gene (NCBI Reference Sequence: NG_015990.1) where base 5375 is a C.

The nucleotide sequence SEQ ID NO: 3 is as
follows:
ACCTCGCACAGCCTCTCCCACAGGTACCATGAAGGTCTCCGCGGCAGCCC
TCGCTGTCATCCTCATTGCTACTGCCCTCTGCGCTCCTGCATCTGCCTCC
CCATGTAAGTCCTGGTCTTGACCACCACAGCCCCTGGAGTCAGACTCTGC
CTAGAATGCCTACCCCATCCCAATGACTGTCCCACACCTTGCCCTCTTTT
CAACTGTAGCATGAATACTGTCTGAACCCCATTATTTACATTCTTATGAA
TTGTCTTACCCATATGTCCTGTTGTCCTTGAGAGATCTCTATGCTGCTTC
ATGGCAGGGATCTCCTGATCAGTTTTTCTGTCTTTAAGGTCTACACCCTC
AAGGCCTACAGGTGTTCACTGAGTGTGGACTCATGTTGGGGAGATTAGTA
GTCATGAGTAGCTCACACTGACTTTGAAACACGTCTGACTCATGCCTGTC
AGCAGCAGGGTTTCTAAGAAATTTAACTAAACTAAAACTTCCTGAGACCC
TGAGACAGCCATATTCTCTCTCATTTCATAAATGTAAAAAAAATTGGCTT
ATGAGAGGGTATGTGGCTTTATTAGAGTGAATACATTTAAAAAACAAATA
AAAACAAGGAGAGGATAATAAAAGCTAATTAAATGGTTACTATGGATCAG
GCATTGTGCTGATTTTGGCACATCTTTTTT.
The nucleotide sequence of SEQ ID NO: 4 is as
follows:
ACCTCGCACAGCCTCTCCCACAGGTACCATGAAGGTCTCCGCGGCAGCCC
TCGCTGTCATCCTCATTGCTACTGCCCTCTGCGCTCCTGCATCTGCCTCC
CCATGTAAGTCCTGGTCTTGACCACCACAGCCCCTGGAGTCAGACTCTGC
CTAGAATGCCTACCCCATCCCAATGACTGTCCCACACCTTGCCCTCTTTT
CAACTGTAGCATGAATACTGTCTGAACCCCATTATTTACATTCTTATGAA
TTGTCTTACCCATATGTCCTGTTGTCCTTGAGAGATCTCTATGCTGCTTC
ATGGCAGGGATCTCCTGATCAGTTTTTCTGTCTTCAAGGTCTACACCCTC
AAGGCCTACAGGTGTTCACTGAGTGTGGACTCATGTTGGGGAGATTAGTA
GTCATGAGTAGCTCACACTGACTTTGAAACACGTCTGACTCATGCCTGTC
AGCAGCAGGGTTTCTAAGAAATTTAACTAAACTAAAACTTCCTGAGACCC
TGAGACAGCCATATTCTCTCTCATTTCATAAATGTAAAAAAAATTGGCTT
ATGAGAGGGTATGTGGCTTTATTAGAGTGAATACATTTAAAAAACAAATA
AAAACAAGGAGAGGATAATAAAAGCTAATTAAATGGTTACTATGGATCAG
GCATTGTGCTGATTTTGGCACATCTTTTTT.

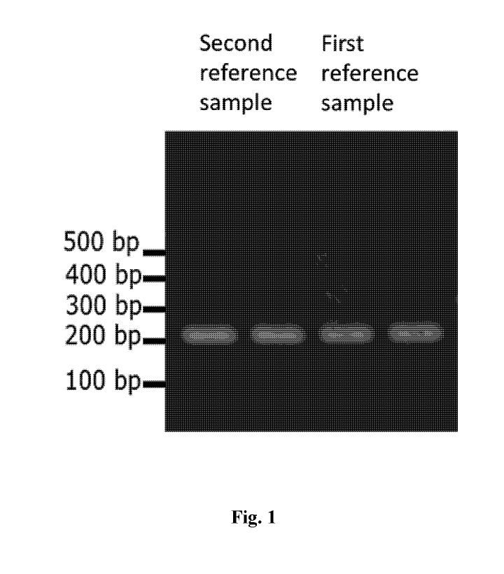

A specific melting curve was observed for each of the first and second reference samples (data not shown), demonstrating the specificity of the primer pair. The PCR products are expected to be 235 bp. The results of gel electrophoresis analysis showed single bands (see FIG. 1), which also demonstrate that the primer pair specifically amplifies the targeted sequence.

### Example 2: RANTES Gene rs2280789 SNP Genotyping

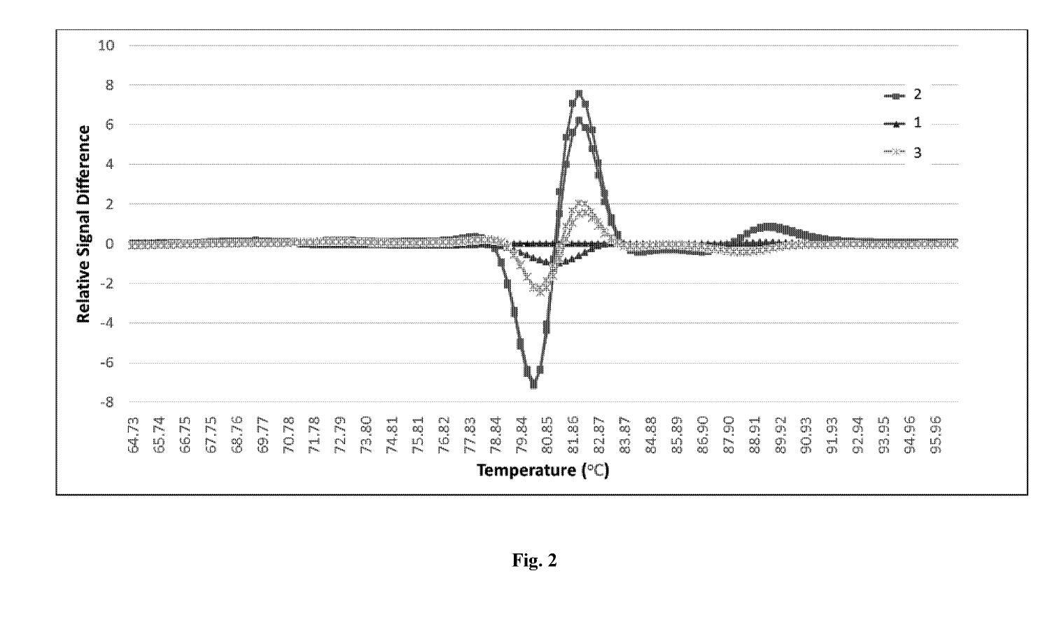

Perform qPCR using the forward primer of SEQ ID NO: 1 and the reverse primer of SEQ ID NO: 2 on a first reference sample and a second reference sample as described in Example 1 and on a genomic DNA sample isolated from mesenchymal stem cells derived from the placenta of a female subject. The experiments were done in triplicate for each sample, and two melting curves were shown for each sample. Calculate the average of the three melting curves of the first reference sample, and subtract it from the three melting curves of the first reference sample, the three melting curves of the second reference sample, and the three melting curves of the genomic DNA sample to obtain three first difference curves, three second difference curves, and three third difference curves, respectively. The results are shown in FIG. 2 (only two difference curves are shown for each group). The first difference curves are represented by 1, the second difference curves are represented by 2, and the third difference curves are represented by 3. As can be seen in FIG. 2, the first and second difference curves each has a specific and characterizing profile, and the third difference curves have a its own specific profile and the profile is in between that of the first difference curves and that of the second difference curves (the profile does not fit better with either one). Accordingly, the genotype of the rs2280789 SNP in the RANTES gene of the genomic DNA sample is determined as TC. The PCR products of the genomic DNA sample were later subjected to Sanger sequencing for validation. The results of Sanger sequencing confirmed that the genotype at the rs2280789 SNP is TC.

### Example 3: Identification of a Greater Risk for Developing Bronchopulmonary Dysplasia (BPD) of a Preterm Infant

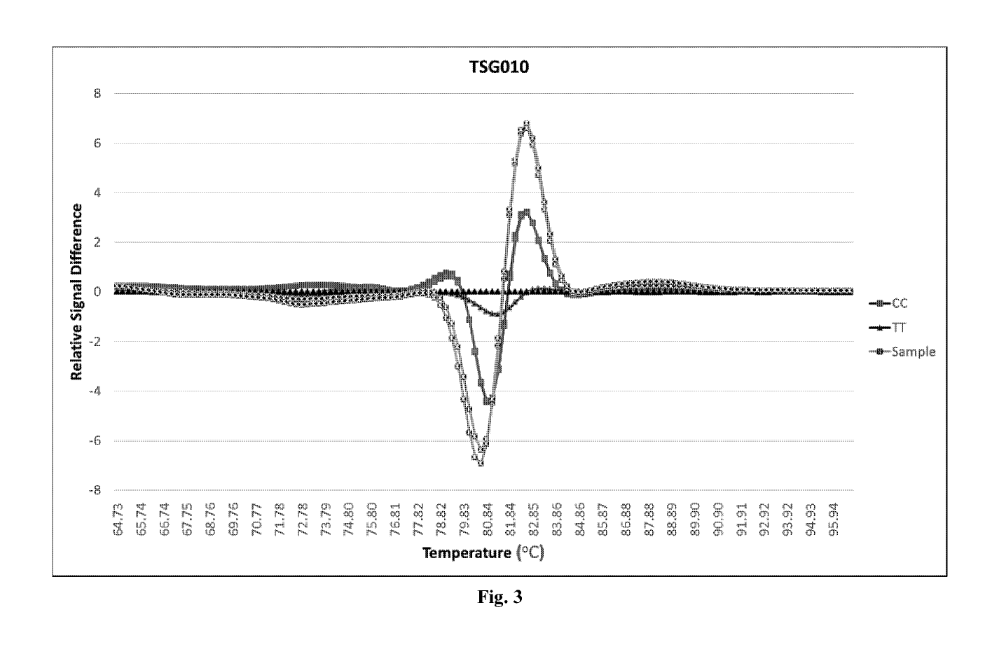

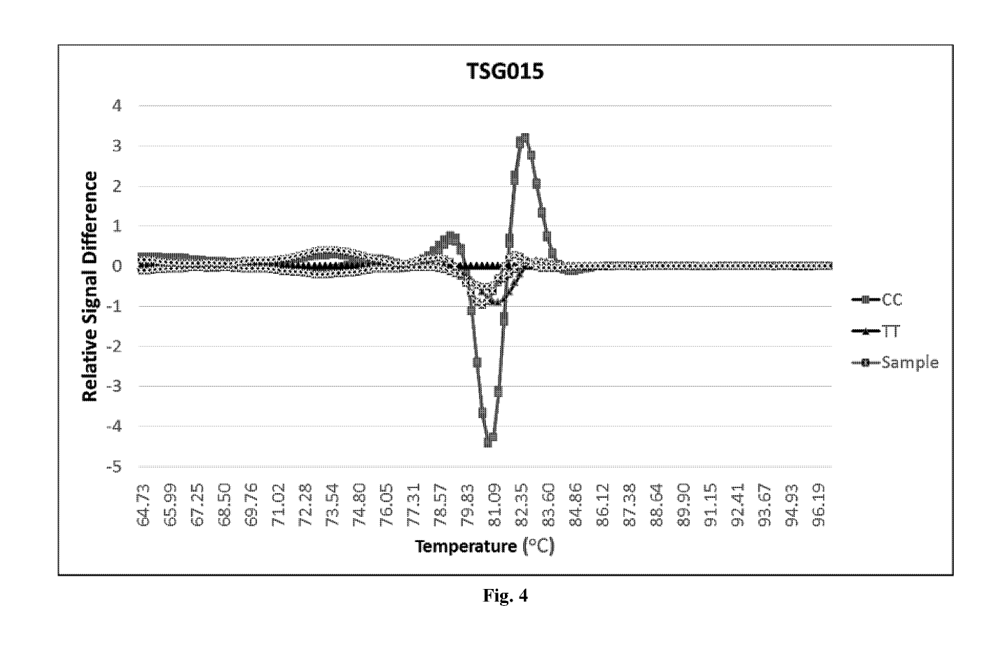

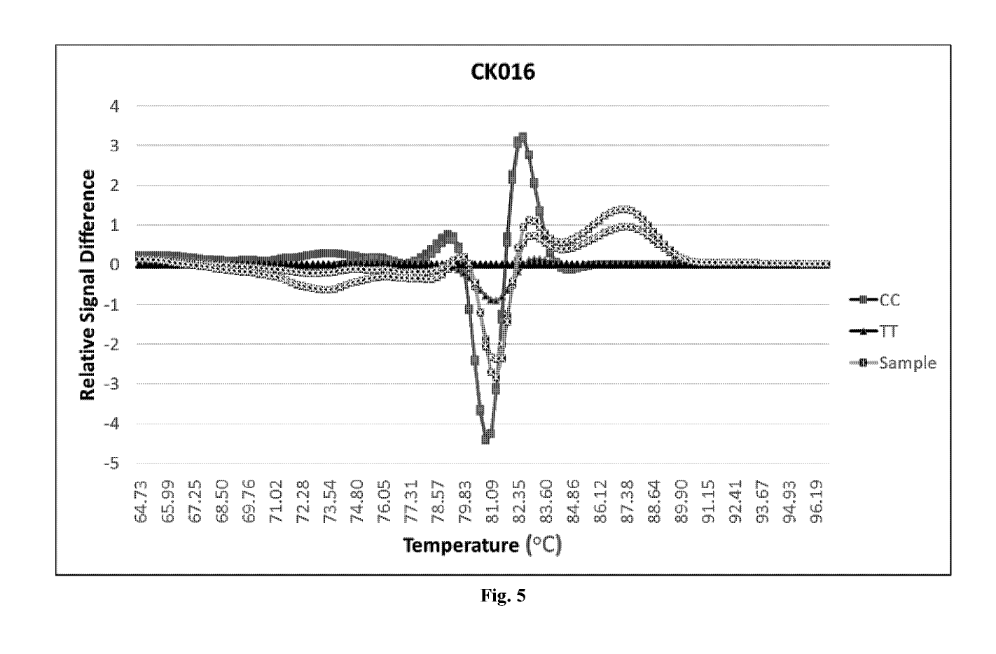

Briefly, the genomic DNAs were isolated from mesenchymal stem cells derived from the placenta of 15 mothers of respective preterm infants. In a parallel test, genomic DNAs were isolated from umbilical cord blood samples (data not shown). The genotype of the rs2280789 SNP in the RANTES gene of each genomic DNA sample was determined through the process as described in Example 2. FIGS. 3-5 are three representative difference curve plots regarding the analysis of respective genomic DNA sample from three subjects (sample name: TSG010, TSG015, and CK016), where the genotyping results were CC, TT and TC, respectively. In FIG. 3, the third difference curves (“Sample”) fit better with the second difference curves (“CC”) than the first difference curves (“TT”), and accordingly, the genotype of the rs2280789 SNP is determined as CC. In FIG. 4, the third difference curves (“Sample”) fit better with the first difference curves (“TT”) than the second difference curves (“CC”), and accordingly, the genotype of the rs2280789 SNP is determined as CC. In FIG. 5, the third difference curves (“Sample”) are in between the first difference curves (“TT”) and the second difference curves (“CC”), and accordingly, the genotype of the rs2280789 SNP is determined as TC. All genotyping results were validated by Sanger sequencing and found to be corrected. Alternatively, the genotype of the rs2280789 SNP in the RANTES gene of each genomic DNA sample was determined based on an arithmetic mean of SP1/RC1 and SP2/RC2, where SP1 and SP2 are the smallest and largest extreme values, respectively, of the average of the third difference curves, and RC1 and RC2 are the smallest and largest extreme values, respectively, of the average of the second difference curves. The genotype was determined as TT if the arithmetic mean was smaller than 0.26, the genotype was determined as TC if the arithmetic mean was in the range of 0.26 to 1.04, and the genotype was determined as TC if the arithmetic mean was larger than 1.04. See Table 1 below.

TABLE 1
Genotyping by arithmetic mean method

| | | | | Sanger | |
|---|---|---|---|---|---|
| Sample | SP1/RC1 | SP2/RC2 | (SP1/RC1 + | se- | Cut-off |
| name | Ratio | Ratio | SP2/RC2)/2 | quencing | value |
| TSG015 | 0.22 | 0.09 | 0.15 | TT | <0.26 |
| LCG011 | 0.04 | 0.32 | 0.18 | TT | <0.26 |
| TSG008* | 0.20 | 0.47 | 0.34 | TC | 0.26-1.04 |
| LCG009* | 0.19 | 0.53 | 0.36 | TC | 0.26-1.04 |
| CK005 | 0.09 | 0.67 | 0.38 | TC | 0.26-1.04 |
| CK016 | 0.54 | 0.30 | 0.42 | TC | 0.26-1.04 |
| CK013 | 0.61 | 0.28 | 0.44 | TC | 0.26-1.04 |
| CK001 | 0.38 | 1.12 | 0.75 | TC | 0.26-1.04 |
| LCG012 | 0.37 | 1.41 | 0.89 | TC | 0.26-1.04 |
| CK004* | 1.03 | 1.36 | 1.19 | CC | >1.04 |
| TSG002* | 0.72 | 1.67 | 1.20 | CC | >1.04 |
| LCG014 | 0.91 | 1.96 | 1.43 | CC | >1.04 |
| CK017 | 1.06 | 2.36 | 1.71 | CC | >1.04 |
| TSG010* | 1.45 | 2.00 | 1.72 | CC | >1.04 |
| LCG006* | 1.56 | 3.19 | 2.37 | CC | >1.04 |

Sample names marked with “*” refer to samples from BPD infants' mothers.

The results of statistical analysis are shown in Table 2 below.

TABLE 2
Identification of a greater risk for developing BPD

| | | Non- | | | P value |
|---|---|---|---|---|---|
| | SNP | BPD | BPD | Chi- | (Chi-square, |
| | type | (n = 9) | (n = 6) | square | one-tailed) |
| Wild-type(%) | TT + TC | 7(77.8) | 2(33.3) | 2.963 | 0.04 |
| Mutant(%) | CC | 2(22.2) | 4(66.7) | | |
| T allele(%) | — | 9(50) | 2(16.67) | 3.445 | 0.03 |
| C allele(%) | — | 9(50) | 10(83.33) | | |

The p values of the Chi-square tests show that there is a statistically significant relationship between the genotype CC or the presence of C allele and BPD.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

### REFERENCES

[1] Rivera L, Siddaiah R, Oji-Mmuo C, Silveyra G R, Silveyra P. Biomarkers for Bronchopulmonary Dysplasia in the Preterm Infant. Frontiers in pediatrics 2016; 4:33.
[2] Yang Y, Qiu J, Kan Q, Zhou X G, Zhou X Y. MicroRNA expression profiling studies on bronchopulmonary dysplasia: a systematic review and meta-analysis. Genetics and molecular research: GMR 2013; 12:5195-206.
[3] Maitre N L, Ballard R A, Ellenberg J H, Davis S D, Greenberg J M, Hamvas A, et al. Respiratory consequences of prematurity: evolution of a diagnosis and development of a comprehensive approach. Journal of perinatology: official journal of the California Perinatal Association 2015; 35:313-21.
[4] Paananen R, Husa A K, Vuolteenaho R, Herva R, Kaukola T, Hallman M. Blood cytokines during the perinatal period in very preterm infants: relationship of inflammatory response and bronchopulmonary dysplasia. The Journal of pediatrics 2009; 154:39-43 e3.
[5] Ambalavanan N, Carlo W A, D'Angio C T, McDonald S A, Das A, Schendel D, et al. Cytokines associated with bronchopulmonary dysplasia or death in extremely low birth weight infants. Pediatrics 2009; 123:1132-41.

1. A method for identifying a greater risk for developing bronchopulmonary dysplasia (BPD) of a preterm human infant, comprising:
obtaining a genomic DNA sample from the preterm infant's mother, genotyping rs2280789 SNP in the RANTES gene using a primer pair comprising a forward primer of SEQ ID NO: 1, and a reverse primer of SEQ ID NO: 2, and
determining the preterm infant as being at risk of developing BPD if the genotype of the rs2280789 SNP is CC,
wherein the genomic DNA sample is derived from placenta or umbilical cord blood, and
wherein the genotype of the rs2280789 SNP is determined by a process comprising:
performing qPCR using the primer pair to obtain a first melting curve of a first reference sample for the genotype TT, a second melting curve of a second reference sample for the genotype CC, and a third melting curve of the genomic DNA sample;
subtracting the first melting curve from each of the first, second and third melting curves to obtain a first, second, and third difference curves, respectively; and
comparing the third difference curve with the first and second difference curves, respectively, so as to determine the genotype of the rs2280789 SNP.

2-3. (canceled)

4. The method of claim 1, wherein the genotype of the rs2280789 SNP is determined as TT if the third difference curve fits better with the first difference curve than the second difference curve; the genotype of the rs2280789 SNP is determined as CC if the third difference curve fits better with the second difference curve than the first difference curve; and if otherwise, the third difference curve is in between the first difference curve and the second difference curve, the genotype of the rs2280789 SNP is determined as TC.

5. The method of claim 1, wherein the genotype of the rs2280789 SNP is determined by a process comprising:
performing qPCR using the primer pair to obtain a plurality of first melting curves of a first reference sample for the genotype TT, a plurality of second melting curves of a second reference sample for the genotype CC, and a plurality of third melting curves of the genomic DNA sample;
subtracting the average of the plurality of first melting curves from each of the plurality of first, second and third melting curves to obtain a plurality of first, second, and third difference curves, respectively; and
comparing the plurality of third difference curves with the plurality of first and second difference curves, respectively, so as to determine the genotype of the rs2280789 SNP.

6. The method of claim 5, wherein the genotype of the rs2280789 SNP is determined as TT if the plurality of third difference curves fits better with the plurality of first difference curves than the plurality of second difference curves; the genotype of the rs2280789 SNP is determined as CC if the plurality of third difference curves fits better with the plurality of second difference curves than the plurality of first difference curves; and if otherwise, the plurality of third difference curves is in between the plurality of first difference curves and the plurality of second difference curves, genotype of the rs2280789 SNP is determined as TC.

7. A primer pair for genotyping rs2280789 SNP in the RANTES gene, comprising a forward primer of SEQ ID NO: 1, and a reverse primer of SEQ ID NO: 2.

8. A method for genotyping rs2280789 SNP in the RANTES gene comprising:
performing qPCR using a forward primer of SEQ ID NO: 1 and a reverse primer of SEQ ID NO: 2 to obtain a first melting curve of a first reference sample for the genotype TT, a second melting curve of a second reference sample for the genotype CC, and a third melting curve of the genomic DNA sample;
subtracting the first melting curve from each of the first, second and third melting curves to obtain a first, second, and third difference curves, respectively; and
comparing the third difference curve with the first and second difference curves, respectively, so as to determine the genotype of the rs2280789 SNP.

9. The method of claim 8, wherein the genotype of the rs2280789 SNP is determined as TT if the third difference curve fits better with the first difference curve than the second difference curve; the genotype of the rs2280789 SNP is determined as CC if the third difference curve fits better with the second difference curve than the first difference curve; and if otherwise, the third difference curve is in between the first difference curve and the second difference curve, the genotype of the rs2280789 SNP is determined as TC.

10. A method for genotyping rs2280789 SNP in the RANTES gene comprising:
performing qPCR using a forward primer of SEQ ID NO: 1 and a reverse primer of SEQ ID NO: 2 to obtain a plurality of first melting curves of a first reference sample for the genotype TT, a plurality of second melting curves of a second reference sample for the genotype CC, and a plurality of third melting curves of the genomic DNA sample;
subtracting the average of the plurality of first melting curves from each of the plurality of first, second and third melting curves to obtain a plurality of first, second, and third difference curves, respectively; and
comparing the plurality of third difference curves with the plurality of first and second difference curves, respectively, so as to determine the genotype of the rs2280789 SNP.

11. The method of claim 10, wherein the genotype of the rs2280789 SNP is determined as TT if the plurality of third difference curves fits better with the plurality of first difference curves than the plurality of second difference curves; the genotype of the rs2280789 SNP is determined as CC if the plurality of third difference curves fits better with the plurality of second difference curves than the plurality of first difference curves; and if otherwise, the plurality of third difference curves is in between the plurality of first difference curves and the plurality of second difference curves, genotype of the rs2280789 SNP is determined as TC.
